(19) United States
(12) Patent Application Publication
(10) Pub. No.: US 2012/0203329 A1
(43) Pub. Date: Aug. 9, 2012

(54) BIFURCATED STENT AND METHOD OF USE

(76) Inventor: Richard R. Heuser, Phoenix, AZ (US)

(21) Appl. No.: 13/022,351

(22) Filed: Feb. 7, 2011

(51) Int. Cl. A61F 2/82

(52) U.S. Cl. 623/1.35

(57) ABSTRACT

A stent system is provided for percutaneous insertion in an artery of a main stent which may include at least one peripheral fenestration defined through the stent wall. The peripheral fenestration may be configured to be expanded in situ to receive a peripheral stent. The stent system also may include a peripheral stent configured to be inserted into the peripheral fenestration of the main stent. The peripheral stent may extend, when inserted in the peripheral fenestration, generally perpendicular to the longitudinal axis of the main stent. The stent system may further include a guidewire, insertable through the peripheral aperture, for maneuvering the main stent into place in the artery. The guidewire may be tapered toward its distal end. The stent system may also include a dilation device for dilating the peripheral stent within the peripheral aperture.

### CROSS REFERENCE TO RELATED APPLICATIONS

The present application is related to U.S. Pat. No. 6,858,038, filed Jun. 21, 2002, entitled “Stent System,” the entire disclosure of which is hereby incorporated by reference in its entirety.

### TECHNICAL FIELD

Embodiments herein relate to the field of percutaneous interventions, and, more specifically, to stents for use in treating aneurysms.

### BACKGROUND

An aneurysm is an abnormal widening or expansion of a blood vessel, such as an artery, which occurs in a localized area of the artery and is typically the result of a weakening of the arterial wall caused by disease. The expansion is usually accompanied by a collection of fluid or clotted blood in the localized area. If the aneurysm is not treated, it typically will continue to expand, and may rupture, causing dangerous internal bleeding.

The most common locations for aneurysms are in the abdominal aorta, between the renal arteries and the split of the abdominal aorta into the left and right common iliac arteries, and in the upper legs, in the common iliac adjacent the take off of the internal iliac. Other vessels can be affected as well. The aneurysms in some cases involve only a single, main artery, but in other cases, one or more secondary arteries, branching from the main artery, are also weakened by disease and abnormally expanded. Such secondary arteries include the renal arteries and the superior mesenteric artery on the abdominal aorta, and the internal iliac off the common iliac artery.

Open surgery has been used to repair aneurysms, but, at least in part due to the morbidity rates associated with open surgery, percutaneous procedures are replacing open surgery. The aneurysm is repaired in the percutaneous procedures by placing a covered stent in the affected main artery. However, such covered stents, particularly in the case of an aneurysm affecting one or more secondary arteries, such as the renal arteries or the internal iliac, may not adequately seal the aneurysm and are prone to leakage in the area adjacent the secondary arteries.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

FIG. 1 illustrates an example of a prior art stent, in accordance with various embodiments;

FIG. 2 illustrates an embodiment of a bifurcated stent in an abdominal aorta adjacent the renal arteries, showing one of two fenestrations being opened for placement of a peripheral stent, in accordance with various embodiments; and

FIG. 3 illustrates the fully assembled bifurcated stent of FIG. 2 once both peripheral stents have been positioned in the takeoffs of the renal arteries, in accordance with various embodiments.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “NB” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous.

Embodiments herein provide bifurcated stents and methods of using bifurcated stents to treat or repair aneurysms, for example, aneurysms and tears in the abdominal aorta, such as those occurring near takeoffs of dependent arteries, such as the renal arteries. In embodiments, the disclosed bifurcated stents may be placed contemporaneously with or independently from the placement of another previously placed or planned endoluminal graft.

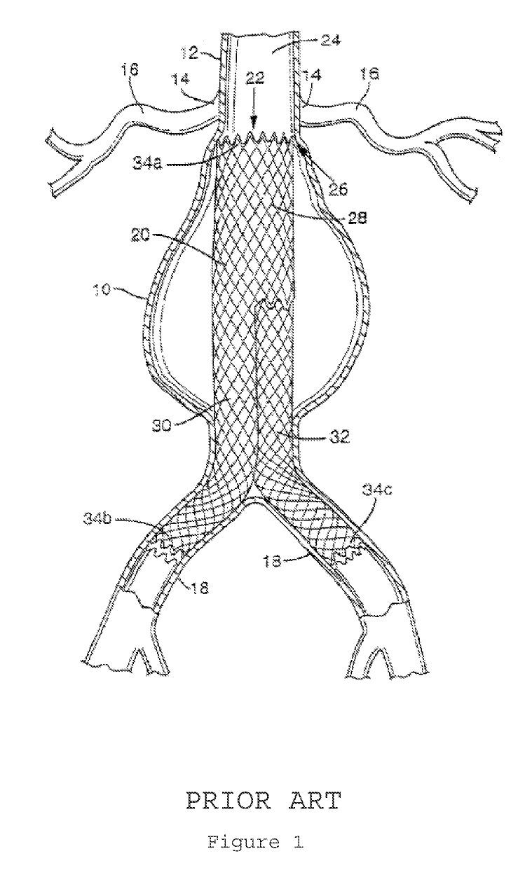

An example of a prior art device for treating an aneurysm 10 in an abdominal aorta 12 is illustrated in FIG. 1. Aneurysm 10 may extend from the takeoffs 14 of the renal arteries 16 down to the split of the abdominal aorta 12 into the left and right common iliacs 18. In the illustrated embodiment, a standard endoluminal graft 20 has been installed in aneurysm 10 in an attempt to provide a flow path 22 for blood 24 past aneurysm 10 and into common iliacs 18. However, because of the involvement in aneurysm 10 of renal arteries 16, graft 20 has not sealed off aneurysm 10 and blood 24 may leak into aneurysm 10 at gap 26. Nonetheless, graft 20 cannot be extended up aorta 12 further without blocking renal arteries 16. Thus, the prior art graft 20 provides no way to treat the aneurysm while maintaining the takeoffs of dependent arteries from the main artery being treated. Occluding the dependent arteries may cause several problems, including allowing the aneurysm to continue filling with blood from collaterals supplying the dependent artery. For an aneurysm on the abdominal aorta, the results can include loss of kidney function, bowel ischemia, perineal ischemia, and impotence.

Prior art graft 20 may include a wide-channel upper portion 28 above an integral narrow-channel portion 30 extending down into one common iliac, and a separately attached narrow-channel portion 32 extending into the other common iliac. Each of the three portions of graft 20 may be constructed of a generally solid and continuous wall 34 a-c wrapped into a cylindrical shape to define a channel with two open ends. The two narrow-channel portions may be coupled, one integrally, the other attached during surgery, to one of the open ends of the wide-channel portion. Each of the portions defines a longitudinal axis, and all three of the axes run generally parallel to one another.

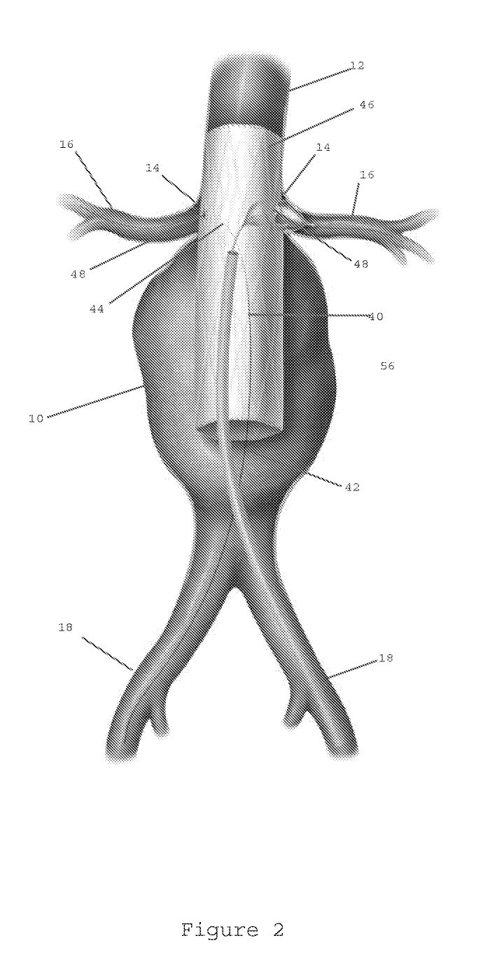

As described, in some abdominal aortic aneurysms, the classic (prior art) endoluminal graft may be difficult to place or may leak because of the short neck of the graft or because of an endoleak (filling of the aneurysm after an endoluminal graft has been placed), for instance due to an inadequate seal on the upper end of the aneurysm. In various embodiments, the bifurcated stents disclosed herein may be used to seal such leaks and/or repair aneurysms extending towards secondary arteries. Thus, disclosed in various embodiments are bifurcated stents that may be used independently, for example, for treating aneurysms or tears in an artery, or they may be used in combination with other grafts, such as the one shown in FIG. 1. As shown in FIG. 2, in various embodiments, two guidewires 40 may be inserted percutaneously, for example, through a single cut down or a large sheath in one groin site, into a common iliac 18, and guided up through the abdominal aorta 12, past the existing graft, if one is present, and into renal arteries 16. In some embodiments, guidewire 40 may include a main body 42, for example, having a diameter of from about 0.03″ to about 0.064″ to provide a sufficiently stiff portion for guiding through the arteries. In some embodiments, guidewire 40 may also include a distal portion 44, and the diameter of guidewire 40 may taper in distal portion 44, for example, over a length of about 8 to 10 cm, and to about half the diameter of the main body, such as to a final diameter of about 0.035″. One of skill in the art will appreciate that the dimensions of these features of guidewire 40 may vary depending on the materials used, the particular artery and percutaneous procedure under consideration, and other factors. In various embodiments, guidewire 40 may be formed from nitinol or a mixture of steel and nitinol, at least in the main body portion. In some embodiments, the distal portion may be formed from a softer, hydrophilic and/or stiffer material, so as to promote entry of the distal portion into the renal arteries, or other dependent artery.

As illustrated in FIG. 2, in various embodiments, with guidewires 40 in place in the artery, a main stent 46 may slide along guidewires 40 for accurate, aligned placement adjacent dependent arteries 16. In various embodiments, main stent 46 is constructed of a wall having a generally cylindrical shape and defining a central longitudinal axis. Main stent 46 may be of either the balloon-expandable or self-expanding type, and may include a flexible covering, for example, of polytetrafluoroethylene (PTFE), and a mesh structure, for example, of nitinol or a nitinol/steel blend. In some embodiments, main stent 46 may be fixedly and removably mounted on another guidewire, such as a balloon catheter, which may be used to slide main stent 46 along guidewires 40. In some embodiments, the mesh structure may be configured to provide for self-expansion of main stent 46.

In some embodiments, main stent 46 may have one or more fenestrations 48 through the wall that are generally opposite one another. As used herein, the word “fenestration” refers to a selectively enlargeable perforation, slit, or other opening in the side wall of main stent 46 that does not include a tube, reinforcing ring, or any other additional hardware. In various embodiments, these two fenestrations 48 may be configured to be aligned with a patient's renal arteries, and the number and configuration of the apertures will depend on the application intended for the stent. In some embodiments, the fenestrations may be slits, holes, or other perforations in main stent 46, which may have, for example, a diameter of about 1 mm. In other embodiments, fenestration may be a single or complex slit, for example, an X-shaped, T-shaped, or star-shaped slit, that may have a length of from about 1 to about 3 mm. In some embodiments, the wall of main stent 46 may be split or thinned in the vicinity of fenestration 48 so that the perforation may be easily enlarged in situ to suit the topography of the main artery and secondary arteries. In various embodiments, such a perforation or slit may allow fenestration 48 to be enlarged as desired without risking the damage to adjacent vascular structures that may occur when the perforation is formed once main stent 46 is in place in the main artery, as may occur with non-perforated prior art stents. In some embodiments, fenestrations 48 may include one or more radiopaque elements that may permit visualization by x-ray or other imaging technologies, and guidewires 40 may be inserted through fenestrations 48 and into dependent arteries 16, for example, to promote accurate longitudinal and rotational positioning of stent 46 so that fenestrations 48 face the dependent arteries 16.

In various embodiments, with main stent 46 in place, adjacent aneurysm 10 and aligned with secondary arteries 16, main stent 46 may be expanded. If stent 46 is self-expanding, a removable restraint, such as a membrane or sheath, may be withdrawn from main stent 46, for example, using the catheter used to push main stent 46 into place. In various embodiments, removal of the restraint may allow the self-expanding structure in main stent 46 to expand and fix main stent 46 in place in the main artery (e.g., abdominal aorta 12). Alternately, if main stent 46 is balloon-expandable, a balloon catheter may be activated to expand main stent 46 and fix it in place in main artery 12.

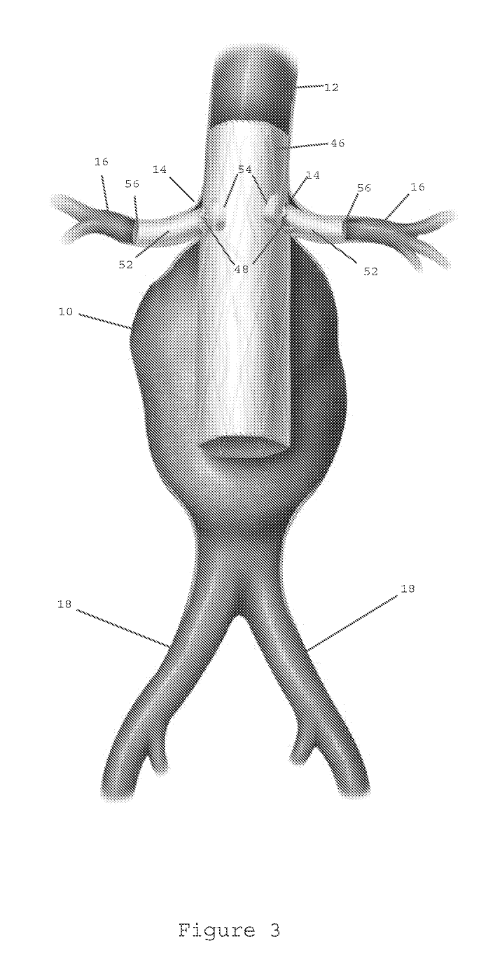

With main stent 46 expanded in place in main artery 12, a standard catheter, such as a JR4 or a Contra catheter, may be used to access one of the secondary arteries 16 (such as a renal artery) via the same sheath used for insertion of main stent 46. In some embodiments, x-ray or other imaging guidance may be employed to ensure proper positioning within radiopaque fenestration 48 and/or secondary artery 16. As illustrated in FIG. 2, once second artery 16 has been accessed, a balloon 50 may be inflated to expand fenestration 48 for placement of peripheral stent 52 therein (see FIG. 3). In some embodiments, peripheral stent 52 may be slid along guidewires 40 and through expanded fenestration 48 to a desired position in secondary artery 16. In various embodiments, peripheral stent 52, which may be a covered, self-expanding or balloon-expandable stent, may include a first end 54 and a second end 56. In some embodiments, peripheral stent 52 may be positioned with first end 54 remaining within main stent 46, and the second end 56 extending out of main stent 46 and into secondary artery 16. The same procedure may then be repeated for placement of the second peripheral stent 52 in the second fenestration 48. In various embodiments, peripheral stent 52 may extend generally perpendicular to longitudinal axis of main stent 46.

In various embodiments, peripheral stents 52 may be moved into place, for example, by inserting and sliding a catheter 56 with a central lumen over guidewire 40. Catheter 56 may also provide a dilation device, such as a balloon, for expanding peripheral stent 52 once it has been positioned in fenestration 48. In some embodiments, the balloon may have two differentially-expanding regions so that first end 54 of peripheral stent 52 may be expanded more than second end 56. For instance, in some examples, first end 54 may be trumpeted or increasingly expanded toward first end 54 such that the proximal portion of peripheral stent 52 may achieve a good seal at the site of the renal outflow. Alternatively, the differential expansion may be accomplished using separate balloons or sequential differential expansion of a single balloon, or by other means. In various embodiments, this differential expansion more firmly fixes peripheral stent 52 in place in fenestration 48 and provides a funnel-shaped conduit to promote blood flow into dependent arteries 16. In various embodiments, the fully-assembled bifurcated stent may have a “T” shape.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. A stent system for installation in a human bodily fluid vessel, the stent system comprising:
a main stent including a generally cylindrical wall defining a central longitudinal axis, the stent including a first fenestration defined through the wall; and
a first peripheral stent configured to be inserted into the first fenestration of the main stent, the first peripheral stent extending, when inserted in the first fenestration, generally perpendicular to the longitudinal axis of the main stent.

2. The stent system of claim 1, wherein the main stent includes a second peripheral fenestration defined through the wall, and further comprising a second peripheral stent configured to be inserted in the second peripheral aperture.

3. The stent system of claim 2, wherein the second peripheral stent, when inserted in the second fenestration, extends generally perpendicular to the longitudinal axis of the main stent.

4. The stent system of claim 1, wherein the tube is formed substantially of a slick material selected to allow the main stent to slide along a guidewire.

5. The stent system of claim 1, wherein the main stent comprises a self-expanding stent.

6. The stent system of claim 5, further comprising a removable restraint disposed around the wall of the main stent.

7. The stent system of claim 6 wherein the removable restraint is configured to break away adjacent the fenestration as the restraint is removed from the main stent.

8. The stent system of claim 1, wherein the main stent comprises a non-self expanding stent.

9. The stent system of claim 8, further comprising a balloon configured to expand the main stent.

10. The stent system of claim 1, wherein the first fenestration comprises an expandable perforation or slit.

11. The stent system of claim 10, wherein a largest dimension of the first fenestration is from 1-3 mm in a non-expanded state.

12. The stent system of claim 10, wherein the first fenestration further comprises an area of thinned mesh.

13. The stent system of claim 2, wherein the first and second fenestrations comprise expandable perforations or slits.

14. The stent system of claim 13, wherein a largest dimension of the first and second fenestrations is from 1-3 mm in a non-expanded state.

15. The stent system of claim 13, wherein the first and second fenestrations further comprise an area of thinned mesh.

16. The stent system of claim 2, wherein the first and second fenestrations comprise one or more radioopacities.

17. The stent system of claim 2, wherein the first and second fenestrations are located approximately 180 degrees apart on opposite walls of the main stent.

18. The stent system of claim 2, wherein the first and second peripheral stents are self-expanding.

19. The stent system of claim 2, wherein the first and second peripheral stents are non-self expanding.

20. The stent system of claim 19, further comprising a balloon configured to expand the first and second peripheral stents.

21. A stent system for installation in a human bodily fluid vessel, the stent system comprising:
a main stent including a generally cylindrical wall defining a central longitudinal axis, the stent including first and second fenestrations defined through the wall and comprising nitinol and PTFE;
a first peripheral stent configured to be inserted into the first fenestration of the main stent; and
a second peripheral stent configured to be inserted in the second peripheral aperture; wherein when inserted in the first and second fenestrations, the first and second peripheral stents extend generally perpendicular to the longitudinal axis of the main stent.

22. The stent system of claim 21, wherein the main stent is self-expanding.

23. The stent system of claim 21, further comprising a guidewire configured to deploy a balloon, wherein the balloon is configured to enlarge the first and second fenestrations.

24. A method of treating an aneurysm in an abdominal aorta in a subject, comprising:
advancing a guidewire through the vasculature to the aneurysm;
advancing a main stent along the guidewire to the aneurysm, wherein the main stent comprises a generally cylindrical wall defining a central longitudinal axis, wherein the stent includes first and second fenestrations defined through the wall;
aligning the first and second fenestrations with first and second renal arteries;
expanding the main stent;
expanding the first fenestration;
inserting a first peripheral stent in the first fenestration;
expanding the s peripheral stent to seal a gap between the first fenestration and the first peripheral stent;
expanding the second fenestration;
inserting a second peripheral stent in the second fenestration; and
expanding the second peripheral stent to seal a gap between the second fenestration and the second peripheral stent.

25. The method of claim 24, wherein expanding the first and second fenestrations comprises expanding a balloon to a desired dimension.
